(19) United States
(12) Patent Application Publication
(10) Pub. No.: US 2008/0132797 A1
(43) Pub. Date: Jun. 5, 2008

(54) MONITORING INFUSION OF A SUBSTANCE

(76) Inventor: Knut BRABRAND, Rasta (NO)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/931,433

(22) Filed: Oct. 31, 2007

Related U.S. Application Data
(63) Continuation-in-part of application No. 10/730,919, filed on Dec. 10, 2003.
(60) Provisional application No. 60/432,256, filed on Dec. 11, 2002.

(30) Foreign Application Priority Data
Dec. 10, 2002 (GB) 0228770.4

(51) Int. Cl. A61B 5/026; A61M 5/142

(52) U.S. Cl. 600/504; 604/151

(57) ABSTRACT

A method of monitoring the intended infusion of a substance into a blood vessel by measuring the flow velocity within the blood vessel downstream of the point of infusion, and providing an indication if an anomaly in the flow velocity occurs.

The present invention relates to an apparatus for and method of monitoring the infusion or injection of a substance into a blood vessel.

There are numerous medical procedures in which it is necessary to infuse or inject a substance into a blood vessel.

Typically an I.V. catheter is inserted into a vein and the substance is fed to this via a flexible tube. The substance may be blood, saline, a drug, a contrast medium, etc. In many cases it is desirable that the infusion occurs slowly and so the substance is simply gravity fed. However, there are circumstances in which it is necessary to force the substance into the blood vessel.

One example is the infusion or injection of a contrast medium used in conjunction with an imaging system such as angiography, computed tomography (CT), ultrasound or MRI. In many applications of these procedures it is necessary to infuse a contrast medium into the part of the body that is to be imaged. The medium must often be infused at a comparatively high rate for effective results to be achieved. As a consequence, in recent years, a number of injector-actuated syringes and power injectors for pressurized injection of contrast medium have been developed.

However, whilst such devices are valuable and effective, they create a risk of extravasation and there is also a risk of other problems with the injection process.

Extravasation is the accidental infusion of fluid such as contrast medium into tissue surrounding a blood vessel, rather than into the blood vessel itself. The causes for extravasation vary. Fragile vasculature or valve disease may cause physiological limitations to the ability of the blood vessel to tolerate the high rate of fluid administration used in some procedures. Extravasation occurs in this scenario through leakage of the injected fluid out of the damaged blood vessel. In computed tomography, for example, contrast injection flow rates can be in the range of 0.1 to 10 ml/s. and so a failure of the vessel may occur. Alternatively, operator error may lead to inappropriate needle placement and patient movement may cause the infusing needle to be pulled from the intended vessel or cause the needle to be pushed through the wall of the vessel. In this scenario, extravasation occurs by fluid passing from the needle directly into the tissue surrounding the blood vessel.

Extravasation of contrast media during intravenous injection is a potential serious complication that might necessitate surgical drainage of the affected region. Even though the incidence rate is low, it is considered to be a major concern and is associated with local pain and possibly necrosis of the tissue. It is therefore important to be able to detect extravasation quickly and reliably so that infusion may then be stopped. If extravasation of a contrast medium occurs during an imaging procedure it is often necessary for the examination to be aborted and repeated at a later stage.

Other infused or injected substances may have more serious effects. For example, chemotherapy drugs can be toxic to tissue if not diluted by blood flow.

Several extravasation detection techniques are known in the art. Two simple and very useful techniques for detecting extravasation are palpation of the patient in the vicinity of the injection site and simple visual observation of the vicinity of the injection site by a trained health care provider.

In the palpation technique, the health care provider manually senses swelling of tissue near the injection resulting from extravasation. By visual observation, it is also sometimes possible to observe directly any swelling of the skin in the vicinity of an injection site resulting from extravasation.

There have been a number of attempts to improve the detection of extravasation. For example, mercury strain gauge plethysmographs measure the volume change resulting from venous blood flow in a cross sectional area of a limb of a patient in order to detect a change in volume of a limb or digit as a result of extravasation.

Photo-plethysmographs measure the optical scattering properties of capillary blood to detect the presence of extravasated fluids in tissue. WO 99/15074 provides a sensor pad having a surface that is placed against a patient. A light source is also provided and a detector on the pad optically detects extravasation by detecting light that is reflected, scattered, etc.

U.S. Pat. No. 4,647,281 discloses subcutaneous temperature sensing of extravasation using a microwave radiometer. The temperature of the subcutaneous tissue where the fluid is injected is compared to that of the injected fluid.

It is also known to detect extravasation by measuring changes in the electrical impedance. Injection fluid in the tissue of the patient also changes the electrical impedance properties of the tissue. Thus, an impedance change of a certain level in the vicinity of the injection site is interpreted as being due to extravasation. WO 99/26686 discloses an electrode patch for attachment to the skin of a patient. It has elongate pick-up electrodes and energizing electrodes. The patch is used to monitor tissue impedance during the procedure and this is compared to a baseline level.

A disadvantage of such devices is that it can be difficult to maintain good electrical contact with the skin of the patient. Also, the location of the patch makes it more difficult to carry out palpation or visual inspection. A similar problem arises with the other prior art detectors. In order to address this problem, U.S. Pat. No. 6,408,204 proposes an apparatus that may be positioned so as not to interfere with palpation or visual inspection. An energy source and a receiver are positioned between a first layer of high dielectric material and a second layer of low dielectric material. If extravasation occurs, as noted above, there is a change in the bulk electrical properties of the tissue. The receiver measures a signal resulting from changes in the energy supplied to the tissue by the energy source.

Other problems that can arise during the course of an injection are an incorrectly positioned needle or I.V. catheter, movement of the patient leading to the needle or I.V. catheter being dislodged or moved to an incorrect position, a blockage or kink within the patient tubing conveying the fluid to be injected or a failure of the power injector. Some of these problems could lead to extravasation as discussed above, but others may simply lead to the fluid not being injected (e.g. in the case of a dislodged I.V. catheter), or fluid flow stopping due to a problem with the tubing or power injector. Although the prevention of effective injection is not a dangerous to the patient as extravasation, it is still important to be able to detect and rectify such problems so ensure efficient patient treatment, especially if the injection is associated with a concurrent scanning procedure using dangerous radiation.

According to the present invention there is provided a method of monitoring the intended infusion of a substance into a blood vessel comprising: measuring the flow velocity within the blood vessel downstream of the point of infusion, and providing an indication if an anomaly in the flow velocity occurs.

Unlike the prior art techniques the present invention is not based on volume changes of tissue induced by extravasation, but on direct monitoring of the increased flow velocity within the blood vessel induced by the infusion. The anomaly may, for example, be an unexpected change in velocity, or it may be a lack of an expected change in velocity.

When fluid is infused into the blood vessel, the flow velocity downstream of the infusion site will increase. Thus, an anomaly in the form of lack of a velocity increase upon initiation of the infusion indicates that extravasation or another problem with the injection has occurred. For example, it may indicate that the needle is not correctly positioned or has become dislodged prior to initiation of the infusion, or that the flow of injected fluid to the patient is not occurring correctly. Since the increase in flow velocity should occur almost immediately infusion commences, this method gives the operator an early warning if a problem occurs.

In addition, the infusion can be monitored as it progresses and problems during the full course of infusion can be identified. If the infusion flow rate remains constant then the fluid flow velocity should also remain constant. Thus, an anomaly in the form of a drop in flow velocity can indicate the onset of extravasation, or the failure of the fluid delivery system.

The method may include measuring a parameter of the injected fluid prior to injection, and providing an indication of an abnormality in the parameter. For example, the injection pressure or flow rate in the power injector could be measured, and a higher or lower value than expected may be used to provide an indication of a problem with the injector equipment. This arrangement allows the operator to assess if a lack of a velocity increase or a drop in flow velocity is due to a failure of the injector mechanism rather than an extravasation or other problem with passage of fluid into the patient. For example, an increased pressure in the power injector could indicate that a pinched or kinked tubing is preventing fluid from reaching the patient. This measurement may be correlated with the Doppler ultrasound measurement of the flow velocity in the blood vessel, which would indicate a reduced velocity compared to the expected velocity. This may trigger an event-specific alarm or notification to the operator. Further, if a sudden pressure decrease in the power injector is correlated with the signal from the ultrasound sensor, and they are in accordance, it would suggest that an extravasation is about to occur, which again could trigger an alarm.

It can be difficult to precisely locate the blood vessel downstream of the point of infusion. Therefore in a preferred embodiment of the invention, an array of detector elements is arranged substantially transverse to the direction of flow of the blood vessel so that at least one element of the array will be located over the vessel. The signal from each detector element varies depending on whether the detector element is located over the blood vessel or over ordinary tissue. This has the advantage that the accuracy with which the detector must be placed is reduced. Provided the detector element is located approximately downstream of the infusion site, at least one element of the array will be located over the vessel, and so it is not necessary to precisely locate the blood vessel at the point of measurement prior to commencing the measurement. Instead, a change in flow velocity will be detected by whichever element or elements of the array are located over the blood vessel.

The change in flow velocity could be detected by measuring the flow velocity at a single point downstream of the point of infusion. In one preferred embodiment, however, the flow velocity within the blood vessel is measured at a plurality of points spaced apart along the extent of the vessel and positioned downstream of the point of infusion. This has the advantage of enabling a user to determine the approximate position within the vessel at which extravasation has occurred.

Although the method of the invention is applicable to any infusion of a substance that causes a detectable increase in blood flow velocity, such as the common scenario of infusion of contrast media at rates of 2 ml/s, it is of particular use where the infusion is at a high rate where the greatest risk of extravasation occurs. The rates of infusion may be over 5 ml/s and sometimes over 10 ml/s. Thus, the invention is of particular application to venous infusions such as contrast agents. The invention may therefore be incorporated as part of a process of generating a medical image.

The invention may be applied only when the infusion is commenced, or when the rate of infusion is increased, these being times when a problem is most likely to occur. However, as noted above, problems may occur during the course of the infusion, for example problems caused by patient movement, and so preferably blood flow velocity changes are continuously or repeatedly monitored during the procedure.

In a simple form of the invention, a change in velocity may be noted by an operator who can then stop the infusion. However, it is preferable that the method further comprises the provision of a notification that an anomaly has been detected and most preferably there may be automatic shut down of the infusion in response to the detection of an anomaly. The method may also include control of an associated scanning procedure when a problem with the infusion is detected. For example, the scanning procedure could be automatically shut down. By automatically halting the scanning procedure the exposure of the patient to radiation can be reduced, and the operator attend to the patient without exposure to radiation, and without needing to deal with the scanner manually.

The invention also extends to an apparatus for monitoring the intended infusion of a substance into a blood vessel comprising: a detector for detecting the flow velocity within the blood vessel downstream of the point of infusion, the apparatus being arranged to provide an output signal when an anomaly in the flow velocity occurs.

The output signal may be a notification such as an alarm. More preferably it comprises a control signal to control the infusion. The output signal need not be “high” to indicate an anomaly. Indeed, it may be preferable to use a fail-safe system in which a “high” output indicates an expected condition, i.e. an expected increase in velocity when extravasation commences, or no change to velocity if there is steady state infusion. Thus, if the “high” signal is lost, either due to extravasation or equipment failure, the infusion can be stopped.

There may a scanning procedure associated with the infusion, and in this case the output signal is preferably a control signal to control the scanning procedure. In particular, the output signal may halt the scanning procedure.

The apparatus may include a sensor for measuring a parameter of the fluid to be injected, i.e. the fluid prior to injection, and a control device arranged to provide an indication of an abnormality in the parameter. The parameter may for example be a pressure or flow rate as discussed above.

Any suitable method of detecting flow velocity may be applied, but preferably ultrasound is employed and it is believed that the most effective technique is ultrasound Doppler. Thus, the detector is preferably an ultrasound Doppler probe which may consist of a single transducer element and more preferably consists of an array of individual transducer elements adapted to be arranged substantially transverse to the direction of flow of the blood vessel so that the position of the blood vessel can be detected and/or the change in flow velocity in the blood vessel can be detected without first knowing the precise location of the blood vessel.

In another embodiment of the invention, a plurality of individual transducer elements (or arrays of transducer elements) are spaced apart along the direction of flow of the blood vessel to form an array (or a two dimensional array) of transducer elements which can track the direction of a blood vessel in use. The probe may be located against the skin of a patient proximate to a vein into which the infusion is being made and downstream of the infusion site. The probe is preferably fixed to the patient's skin using an adhesive. In accordance with standard practice, a coupling medium (ultrasound gel) should preferably be applied to the patient's skin under the transducer elements.

The probe may be connected to a display unit in the conventional manner in which case increases in flow velocity will be visible conventionally as bright patches on the display. These may be detected using conventional techniques, for example by comparing pixel brightness in a preselected region on the display.

Alternatively, the display can be dispensed with and a direct indication of velocity produced. Normally the velocity of the infusion will be significantly higher than any other velocity of flow in the region concerned and so precise measurement is not required.

An analogue signal voltage which is generally proportional to the detected flow velocity may be provided as the output from the detector. This could be used to drive a simply calibrated meter. Additionally or alternatively the voltage may be compared to a threshold voltage such that when this is exceeded an indication is provided that extravasation has (or has not) occurred.

In many applications it may be preferable to use a digital system. If the output from the detector is not in digital form then it may be converted using a conventional analogue-digital converter. The output may then be fed to a processor such as a personal computer or a custom processor incorporated into the apparatus.

Regardless of the system used, an output control signal may then be provided to control the infusion pump. In a simple form this may operate a relay to cut the power to the pump, or if the pump is computer controlled it may be a digital control signal. Preferably, this is arranged on a fail-safe basis as discussed above. For example, it may be arranged so that the pump will only operate if a “high” signal is received from the apparatus of the invention.

Alternatively, a valve arrangement may be used to prevent flow to the vein.

It will be appreciated that the invention extends to a system for giving an infusion comprising an infusion pump arranged to infuse a substance into a blood vessel and an extravasation detector according to the apparatus defined above wherein the detector apparatus is arranged to control the infusion pump. The invention also extends to a method of giving such an infusion comprising the use of such apparatus.

Certain embodiments of the invention will now be described, by way of example only, and with reference to the accompanying drawings in which:—

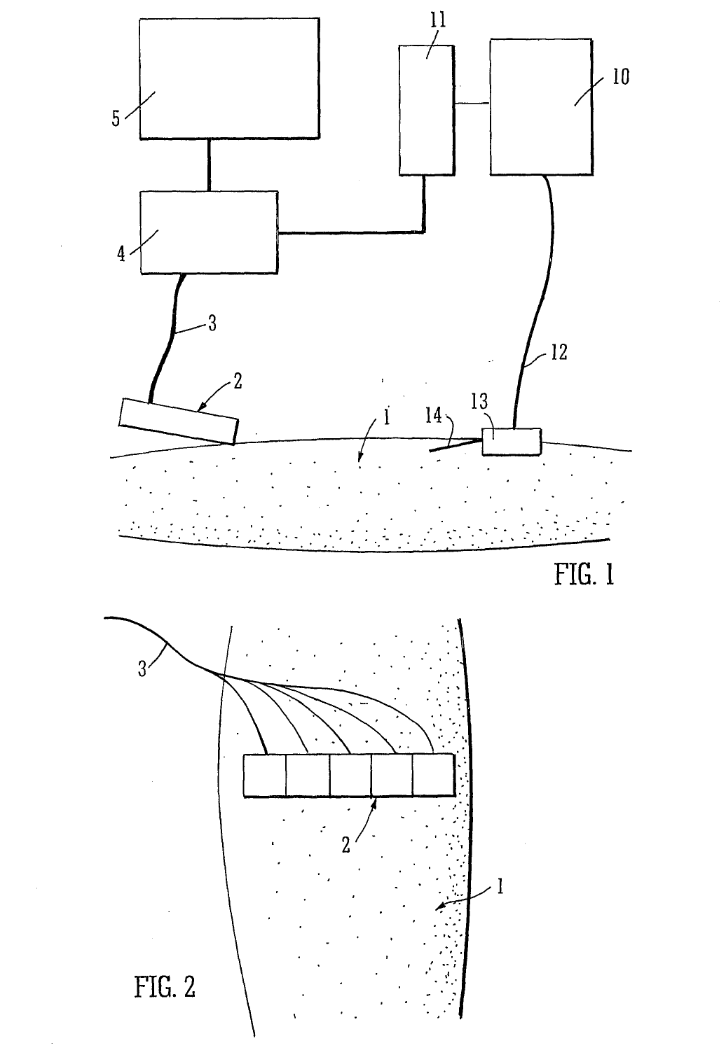

FIG. 1 is a schematic view of a first embodiment of the invention;

FIG. 2 is a schematic view of a modified version of the FIG. 1 embodiment;

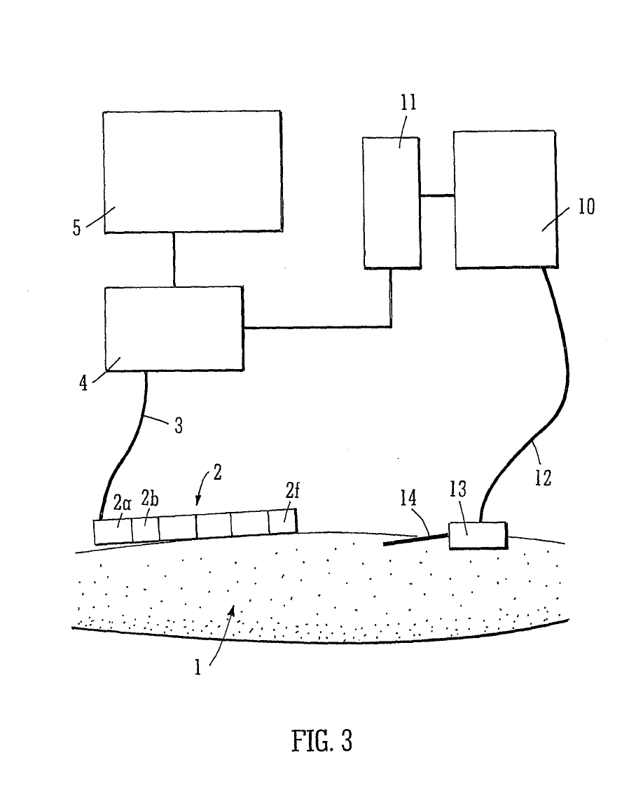

FIG. 3 is a schematic view of an alternative embodiment of the invention;

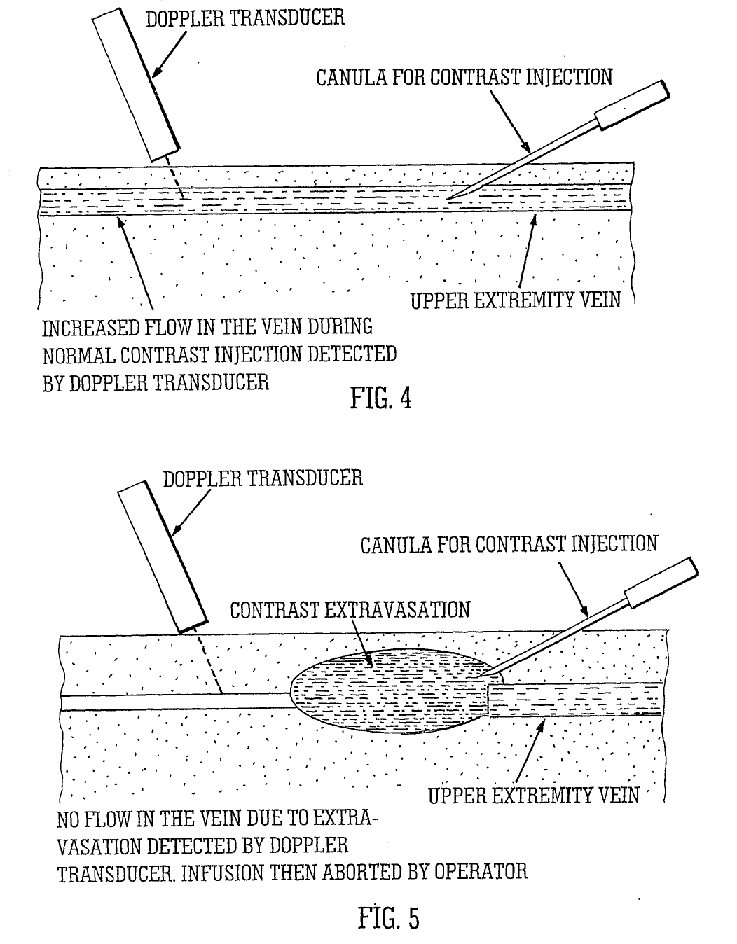

FIG. 4 is a diagram illustrating the use of the embodiment of FIG. 1 where no extravasation has occurred; and

FIG. 5 is a diagram illustrating the use of the embodiment of FIG. 1 where extravasation has occurred.

In FIG. 1 a patient's arm is illustrated at 1. A contrast medium is being infused into the patient from a pump 10. The pump is controlled by an electronic pump controller 11, which varies the pump speed as required and starts and stops it.

The contrast medium flows via flexible tube 12 to I.V. catheter arrangement 13, which comprises a connector for connection to the flexible tube, and a fine bore tube 14 which has been inserted into a vein in the known manner.

Ultrasound Doppler probe 2 is placed above the same vein and a convenient distance downstream so as to be clear of the infusion site. The Doppler probe consists of a single transducer element 2 which in use is placed at an angle to the vein to create and detect a Doppler shift from the flow. The probe 2 is connected via a flexible lead 3 to a processor unit 4. This converts the output from the probe 2 into a form that may be displayed as an image on display unit 5 in the conventional manner. In addition it provides a digital signal proportional to the flow velocity detected by the probe 2. This value is then also displayed on display 5.

The ultrasound probe should use a suitable frequency to provide a good reading of the blood flow velocity. Studies of ultrasound transducers have shown that ultrasound frequencies between 20 kHz and 100 MHz can be used. Preferably, a frequency in the range of 2 MHz to 10 MHz, and, even more preferably, in the range of 4 MHz to 7 MHz, are used, as these have been found to provide good functionality.

In order to obtain a reading of flow velocity in the blood vessel there needs to be a component of the movement of fluid along the line of the emitted ultrasound. As noted above, the ultrasound probe is placed at an angle to the vein to achieve this. In order to obtain a signal the angle cannot be a right angle, and the presence of body tissue and skin between the vein and the probe means that the probe cannot be parallel to the flow direction. Preferably the probe is placed at an angle between 30 and 60 degrees to the vein or skin surface, and, even more preferably, between approximately 40 and approximately 50 degrees.

After the ultrasound reading is taken, the unit 4 determines whether the velocity corresponds to a flow of contrast medium along the vein.

The processor unit 4 and controller 11 together form a control device that receives data concerning flow velocity and injection parameters, and provides a signal to the power injector 10 and a scanner as required in order to control the injection and an associated scanning procedure. It will be appreciated that various arrangements could be used to implement the required control device that monitors and controls the injection process. There can also be data passed back to the control device from the power injector, such as data from a pressure sensor, which can be used to indicate if there is a potential problem with the power injector or other injection.

When the infusion is to commence, the operator sets the desired infusion rate by inputting it into the pump controller 11 and then inputs a start signal into unit 4 by pressing a key (not shown). This in turn transmits a start signal to the pump controller 11 which energises the pump and causes it to run at the desired speed.

The processor unit 4 then checks the flow velocity as described above. If it is not satisfactory within a pre-determined short period of time, i.e. if a velocity increase corresponding to the expected increase from the infusion flow rate does not occur, then the infusion will be stopped. Monitoring of the infusion then continues, and if the velocity drops by more than a set limit during steady state infusion then the unit can provide an alert and/or stop the infusion.

The set limit for the drop in flow velocity may be a default value, or it may be input into the apparatus by the operator dependent upon the particular injection regime. The set limit could be as high as 100%, but preferably it is lower in order to allow the system to be triggered by partial failure in the injection process. For example, a drop of 50% or 25% of the steady state Doppler signal strength could be used to trigger the second signal. In the case of a 25% drop, if the steady state signal was 40 dB above the signal strength when measuring the vein with no injection, then the second signal would be triggered if the signal strength dropped by 10 dB.

As may be seen from FIG. 4, if the I.V. catheter is properly placed and the contrast medium flows as desired along the vein, this will lead to an increased flow velocity in the vein. This is detected by ultrasound probe 2 and, as described above, the processor unit 4 will therefore determine that no extravasation or other problem has occurred. It will therefore continue to send a “pump” signal to pump controller 11.

FIG. 5 shows the situation that might occur when there is extravasation of contrast medium and consequently no flow in the vein. This results in a low or zero velocity output from the probe 2 from which the processor unit 4 determines that extravasation has occurred due to a lack of a velocity increase. It therefore immediately sends a “stop” signal to pump controller 11 which stops pump 10. In this way, the infusion may be stopped almost as soon as the problem occurs with the result that only a small amount of contrast medium enters the tissue surrounding the vein.

Although the situation illustrated in FIG. 5 is most likely to occur when the infusion commences, the processor unit constantly monitors the output from the probe 2 throughout the infusion procedure and can stop the pump at any time, for example if the flow velocity drops as discussed above.

FIG. 2 shows a modified version of the embodiment of FIG. 1 in which an array of individual transducer elements is provided and in use is arranged on the patient's arm substantially transverse to the direction of flow of the vein (i.e. normal to the plane of FIG. 1). Thus, the precise location of the vein need not be known prior to measurement. The signal from each transducer varies depending upon whether it is situated above tissue or above a vein (a Doppler shift will be detected if the transducer is directed towards moving fluid such as blood flowing in a vein). By monitoring the signals received by each transducer element in the array, the location of the vein can be detected. Once it has been determined which transducer elements are situated over the vein, those transducer elements can be monitored for changes in the flow velocity within the vein and hence it can be determined whether or not extravasation has occurred.

FIG. 3 shows an alternative embodiment of the invention in which the Doppler probe 2 consists of a number of individual transducer elements 2a-2f. These transducer elements are spaced at regular intervals to form an array which can be placed on a patient's arm downstream of the I.V. catheter arrangement 13 to extend along the vein in the flow direction.

In a modified version of the embodiment of FIG. 3 (not illustrated), a two dimensional array of individual transducer elements is provided such that the elements extend both substantially transverse and substantially parallel to the direction of flow of the vein. In this way, the precise location of the vein need not be known at each measurement point before commencing measurement.

Instead, as previously described in relation to FIG. 2, it is determined which elements of the array are situated over the vein and those elements are monitored for changes in the flow velocity within the vein. Each transducer element that is over the vein measures the flow rate at a respective point along the vein and this information is provided to the processor unit 4. The processor can therefore determine the approximate position along the vein at which extravasation has occurred. Thus for example, if the flow velocity measured at elements 2a to 2c corresponds to the flow velocity of the contrast medium within the vein but the velocity measured at element 2d does not, the processor determines that extravasation has occurred in the region of transducer element 2d. The remaining parts shown in FIG. 3 correspond to those shown in FIG. 1 and so are not described again here.

It should be noted that although extravasation is the problem described in relation to the Figures, the invention is not restricted to the detection of extravasation, and instead the anomaly identified by the monitoring apparatus can be used to alert the operator to other problems, such as injector equipment failure. In addition, whilst the preferred embodiments refer to the injection of contrast medium, it will be appreciated from the discussion above that the invention is not limited to this type of fluid, but has applications in monitoring the injection of other types of fluids as well, for example in chemotherapy injections and so on.

1. A method of monitoring the intended infusion of a substance into a blood vessel comprising: measuring the flow velocity within the blood vessel downstream of the point of infusion, and providing an indication if an anomaly in the flow velocity occurs.

2. A method as claimed in claim 1, wherein an array of detector elements is arranged substantially transverse to the direction of flow of the blood vessel downstream of the point of infusion so as to locate at least one said detector element over the vessel.

3. A method as claimed in claim 1, wherein the flow velocity within the blood vessel is measured at a plurality of points spaced apart along the vessel and downstream of the point of infusion.

4. A method as claimed in claim 1, wherein the infusion is a venous infusion of a contrast medium.

5. A method as claimed in claim 1, wherein flow velocity changes are continuously or repeatedly monitored during the procedure.

6. A method as claimed in claim 1, further comprising the automatic shutdown of the infusion in response to the detection of an anomaly.

7. A method as claimed in claim 1, wherein the anomaly comprises a lack of flow velocity increase when infusion commences.

8. A method as claimed in claim 1, wherein the anomaly comprises a flow velocity decrease during steady state infusion.

9. A method as claimed in claim 1, wherein the infusion is associated with a scanning procedure and the method comprises control of the scanning procedure in response to the detection of an anomaly.

10. A method as claimed in claim 1, comprising measuring a parameter of the injected fluid prior to injection, and providing an indication of an abnormality in the parameter.

11. An apparatus for monitoring the intended infusion of a substance into a blood vessel comprising a detector for detecting the flow velocity within the blood vessel downstream of the point of infusion, the apparatus being arranged to provide an output signal when an anomaly in the flow velocity occurs.

12. An apparatus as claimed in claim 11, wherein the detector comprises an array of detector elements that may be arranged substantially transverse to the direction of flow of the blood vessel so as to locate at least one said detector element over the vessel.

13. An apparatus as claimed in claim 11, the detector being adapted to measure the flow velocity within the blood vessel at a plurality of points spaced apart along the vessel and downstream of the point of infusion.

14. An apparatus as claimed in claim 11, wherein the output signal indicates an anomaly in the form of a lack of flow velocity increase when infusion commences.

15. An apparatus as claimed in claim 11, wherein the output signal indicates a flow velocity decrease during steady state infusion.

16. An apparatus as claimed in claim 11, wherein the output signal is a control signal to control the infusion.

17. An apparatus as claimed in claim 11, wherein there is a scanning procedure associated with the infusion, and the output signal is a control signal to control the scanning procedure.

18. An apparatus as claimed in claim 11, comprising a sensor for measuring a parameter of the fluid to be injected wherein the apparatus is arranged to provide an indication of an abnormality in the parameter.

19. A system for giving an infusion comprising an infusion pump arranged to infuse a substance into a blood vessel and an apparatus according to claim 11, wherein the apparatus is arranged to control the infusion pump.
